(19) United States
(12) Patent Application Publication
(10) Pub. No.: US 2019/0046786 A1
(43) Pub. Date: Feb. 14, 2019

(54) ELECTRIC COSMETIC ARTICLE HAVING A REPOSITIONABLE BATTERY

(71) Applicant: L'OREAL, Paris (FR)

(72) Inventor: Thi Hong Lien PLANARD-LUONG, Chevilly La Rue (FR)

(73) Assignee: L'OREAL, Paris (FR)

(21) Appl. No.: 16/081,616

(22) PCT Filed: Mar. 3, 2017
(86) PCT No.: PCT/FR2017/050483

§ 371 Date: Aug. 31, 2018

(30) Foreign Application Priority Data
Mar. 4, 2016 (FR) 1651827

(51) Int. Cl. A61N 1/04; A61N 1/30; A61N 1/32; A61N 1/36

(52) U.S. Cl. CPC A61N 1/0428; A61N 1/0412; A61N 1/36014; A61N 1/325; A61N 1/30

(57) ABSTRACT

The present application relates to a flexible cosmetic article comprising at least one substrate (50) on which at least a first pair of electrodes (2,3) and a one-piece unit (12) are placed, the one-piece unit (12) comprising at least one electric power source (1,11) as well as a repositionable adhesive material (5) on at least one portion of its outer surface (33) facing the first pair of electrodes (2,3).

The present invention relates to articles for cosmetically treating human keratin materials with an electrical current.

In the context of the present invention, the term “article” is understood to mean a mask, a patch, a pad, a strip or a bandelet capable of being applied to human keratin materials.

The expression “cosmetic product” is understood to mean any composition such as defined in Council Directive 93/35/EEC of Jun. 14, 1993.

Throughout this text, the expression “including a” must be interpreted as being synonymous with “including at least one”.

### BACKGROUND

Passive masks for applying cosmetic compositions are known. Document U.S. Pat. No. 6,702,792 for example describes a cellulose mask impregnated with a facial lotion.

However, the quantity of molecules administrated using passive techniques remains small. In order to improve this aspect, active masks or patches have been developed that implement electrodes connected to a power source.

Thus it is known to treat human keratin materials using iontophoresis devices (J. Singh, K. S. Bhatia, topical iontophoretic drug delivery: pathways, principles, factors and skin irritation, Med. Res. Rev., vol 16, No. 3, 285-296, 1996).

Iontophoresis allows active agents to diffuse through the skin by virtue of an electrical stimulation in an non-invasive manner. The current administered may be adjustable in magnitude and in direction (anodal or cathodal). The transcutaneous diffusion of molecules via iontophoresis is based on two principles: electro-repulsion and electro-osmosis.

Electro-repulsion is the migration of an ionized molecule by repulsion of charges of the same sign. Thus, if a substance is positively charged, it will diffuse through the skin at the anode (+).

Electro-osmosis is the migration of a molecule, even though it is not ionized, via entrainment related to the flow of water from the anode to the cathode during the iontophoresis. The migration is in particular due to the negative charge of the skin. Under the effect of a current, the water or a solvent entrains, during their migration, dissolved substances.

Iontophoresis technology is highly used to increase the penetration of drugs or of cosmetic active agents. Apart from formulation-related chemical factors and skin-related biological factors, penetration performance is dependent on the magnitude of the current and on the application time.

In order to increase the effectiveness of the iontophoresis, it is necessary to develop specific devices that optimize the penetration of active agents through the skin.

It is known to use iontophoresis patches or masks.

U.S. Pat. No. 6,157,858 or 7,069,088 for example describe masks including at least one pair of electrodes. The electrodes are placed on a malleable carrier. They are supplied by a generator external to the carrier located within a casing. Before the article is put in place on a chosen bodily zone, a cosmetic composition is applied to this zone. The article is then put in place on the face and the electrodes are supplied with electrical current. The duration of the treatment varies between 15 minutes and 1 hour.

Iontophoresis articles may be supplied by a source of electrical power placed on the mask. Such an arrangement is for example described in document FR 2 960 788. The integration of the source of electrical power of the mask may allow use of electrical wires connecting the mask to an exterior source to be avoided. The comfort of use of the mask is then increased and it becomes easier for the user to use.

At the present time, the source of electrical power is fastened to the mask irremovably. It is necessary to have one source of electrical power per mask. Thus, if the mask has been used and must be thrown away, the source of electrical power is thrown away at the same time as the mask even though it is often still usable. Likewise, if a user possesses a plurality of masks, she possesses as many batteries as masks whereas a single battery would suffice to supply all the masks. Likewise again, once the battery has been placed on the mask, the treatment is set. The consumer cannot invert the polarities of the battery in order to change treatment, for example in order to pass from a skin-care treatment to a skin-cleaning treatment. To do this, she needs two masks with differently positioned batteries.

The waste of batteries of iontophoresis articles is currently quite considerable.

There is a need to provide iontophoresis articles meeting the new requirements of respect of the planet and durable development.

There is a need to provide such articles that are more ecological and, possibly, biodegradable.

There is a need to provide such articles that are inexpensive and simple to manufacture.

The article must furthermore be easy to manipulate.

The invention aims to solve all or some of the aforementioned requirements and to further improve articles for cosmetically treating human keratin materials with an electric current.

One subject of the invention is a supple cosmetic article comprising at least one carrier on which are placed at least one first pair of electrodes comprising a first electrode and a first counter-electrode and a unitary block including at least one source of electrical power, the unitary block is connected without cable to the first pair of electrodes and the source of electrical power comprises a first terminal that is connected to the first electrode and a second terminal that is connected to the first counter-electrode, the unitary block including a repositionable adhesive material on at least one segment of its exterior surface, said segment being located facing the first pair of electrodes.

By virtue of the presence of the repositionable adhesive on the unitary block, it may be detached from the first pair of electrodes of a first article according to the invention and replaced:
either on the same first article on:
   the first pair of electrodes without inverting the terminals,
   the same first pair of electrodes while inverting the terminals,
   a second pair of electrodes,
or on a second pair of electrodes of a second article according to the invention.

The article according to the invention thus includes a multi-use unitary block that may be used either a plurality of times for the same mask, or sometimes for one mask, sometimes for one or more other masks.

The article according to the invention is ecological in that it allows the amount of wasted electrical power sources, in particular batteries, to be decreased. It is consistent with the present-day requirements of ecology and of durable development.

The manipulation of the unitary block is easy for the consumer.

Savings are made.

In addition, once the battery has been detached from the mask by virtue of the repositionable adhesive, the discharge of the latter is decreased. It may be used for longer.

The invention also relates to a cosmetic method comprising the steps consisting in:
putting in place on a bodily zone, in particular on the face, a first article, in order to carry out a first cosmetic treatment,
making a first electrical current flow within said first article thus put in place,
removing the unitary block,
repositioning the removed unitary block on a second pair of electrodes forming part either of the first article or of a second article, in order to carry out a second cosmetic treatment,
making a second electrical current flow within said first or second article thus put in place.

According to a first embodiment of the method according to the invention, the first cosmetic treatment is different from the second cosmetic treatment.

According to a second embodiment of the method according to the invention, the first cosmetic treatment is identical to the second cosmetic treatment.

Preferably, the first cosmetic treatment and the second cosmetic treatment are chosen from a make-up treatment, a care treatment, a cleaning treatment or an extracting treatment.

### GENERAL DEFINITIONS

“Connected without cable” means connected without electrical cable, nor electrical wire.

An “electrode” is placed on a conductive carrier.

According to the invention, the term “electrode” is understood to mean a positively charged electrode (anode) or negatively charged electrode (cathode). This electrode is generally placed on the external surface of the article in order to make direct contact with the keratin materials. However, the electrode may also be inserted into the external wall of the article. In this case, it does not make direct contact with the keratin materials but makes contact therewith via a cosmetic composition and its carrier. In general, the electrode makes contact with the zone to be treated.

An electrode may for example take the form of a sphere or pad for example or of a supple film printed in conductive ink. An electrode may form an array of conductive areas that are connected together and to one polarity of the power source.

The expression “counter-electrode” is understood to mean a negatively charged electrode (cathode) or positively charged electrode (anode). The charge of the counter-electrode is opposite to that of the electrode. The polarity of an electrode may be modified. The electrode is generally active, contrary to the counter-electrode, which is not necessarily located in the zone to be treated.

A “pair of electrodes” is formed by an electrode and a counter-electrode.

The expression “electrical supply system” is understood to mean an electrical assembly that is able to induce a potential difference between the electrode and the counter-electrode.

The term “compartment” is understood to mean a division produced in an article in order to house cosmetic compositions that generally differ from one compartment to the next. Each compartment includes at least one pair of electrodes. An example article comprising a plurality of compartments is described in patent application WO 2015/118246.

A “repositionable adhesive” is an adhesive that is tacky enough that if the unitary block is placed on a first pair of electrodes of a first article, the unitary block is held in place throughout the treatment then can be removed without being damaged, nor damaging the first pair of electrodes. An example of a repositionable adhesive usable according to the invention is a PSA (pressure sensitive adhesive) including acrylate, methacrylate or silicon-containing groups. PSAs are described in documents WO1994000525 or WO2009035765 belonging to the company 3M for example.

Preferably, the repositionable adhesive has a tack (or tackability) of at least 200 g measured according to standard ASTM D2979 with a Polyken™ probe tack tester.

Even more preferably, the repositionable adhesive has a peel force of at least 100 N/m measured according to standard ASTM D 1000.

A “supple article” is an article that is flexible and deformable. It preferably has a Shore A hardness lower than 25, preferably comprised between 1 to 20, and more preferably comprised between 10 and 15.

### PREFERRED EMBODIMENTS

The assembly according to the invention may comprise one or more of the following features, implemented alone or in any technically possible combination:
The repositionable adhesive material at least partially encircles the first terminal and the second terminal. The attachment is improved and the current is more uniform.
The repositionable adhesive material is a viscoelastic silicone gel. This material is effective. Other materials than silicone may be used.
The repositionable material forms a layer of thickness comprised between one hundredth of a millimeter and 5 millimeters. This thickness is optimal for a good hold of the unitary block.
The unitary block is at least partially supple. The suppleness of the article is improved.
The source of electrical power may provide a potential difference higher than or equal to 1 V. This potential range is suitable to an electrical treatment of the skin.
The source of electrical power is a source of DC voltage. The treatment is more effective and faster.
The source of electrical power comprises one or more button cells or one or more double button cells. The unitary block is miniature and easy to manipulate.
The carrier comprises an extension.
The carrier is impregnated with a composition.
The article comprises a plurality of compartments each comprising a composition. Various treatments may take place, depending on the region of the face for example.

### ELECTRICAL PARAMETERS

The source of electrical power may include any battery or any accumulator. The potential difference between the electrodes is for example comprised between 1.2 V and 24 V, and preferably between 1.2 and 10 V. Where appropriate, the passage of the current may create point heating.

At equivalent current density, the article may in particular deliver a current density, at the skin, preferably lower than or equal to 0.500 mA/cm², for example comprised between 0.001 mA/cm² and 0.500 mA/cm², for example between 0.001 mA/cm² and 0.10 mA/cm².

### ELECTRODES

The electrodes may have an apparent free surface allowing them to make direct contact with the skin.

At least one electrode may, for example, include:
a composite material (plastic filled with carbon microfibers),
a conductive fabric,
a conductive nonwoven,
a polymer material made conductive,
a fibrous material,
conductive polymeric fibers, for example such as described in publication CN101532190,
carbon fibers, for example such as described in publication JP2009179915,
silicones made conductive by addition of conductive fillers such as silver, copper or carbon. Such silicones are, for example, sold by the companies Saint Gobain, Plastics Performance and Aquitaine Caoutchouc 2000,
conductive metal fabrics, for example sold by the companies Utexbel and Cousin Biotech,
carbon-containing vinyl, for example sold by the companies Copema and Rexam,
electrosurgical pads, for example sold by the companies Copema and 3M,
intrinsically conductive polymers, for example sold by the company Paniplast.

### COSMETIC COMPOSITION

The cosmetic composition may be deposited directly on the zone to be treated by the user.

The cosmetic composition may also be impregnated or dispersed in the article.

The active ingredient is, preferably, charged. By “charged”, what is meant is any active ingredient present at least partially in ionic form the ions of which possess a net charge that is either positive, or negative, able to ensure their mobility within the composition under the effect of an electric field. Thus, the active agent is subjected directly to the attraction or the repulsion of the electrodes.

Advantageously, the cosmetic composition is chosen among care compositions, washing compositions, purifying compositions, exfoliating compositions, desquamating compositions, massaging compositions, thinning compositions, make-up compositions, make-up-removal compositions, cleaning compositions or whitening compositions.

More advantageously, the cosmetic composition takes the form of an aqueous solution, an oil, an emulsion, a powder or a gel.

Whatever the embodiment in question, the article may exert an action on the skin by iontophoresis and/or electro-osmosis.

The composition may include a composition for activating an inactivated active ingredient present within the article, for example in freeze-dried form. In this case, the composition may be devoid of a charged active ingredient. The composition may include a solvent having positively and negatively charged species, for example an ionic aqueous solution or deionized water or even an NaCl solution.

It is also possible for the user to apply an activating composition, for example a solvent, to the article. For example, he may apply running water, whereas the latter is not provided within a given packaging with the article.

To bring the article and the activating composition into contact, the user may pour the composition onto the article. The latter is, for example, present in a wallet or tub allowing the composition to be poured thereon. As a variant, the user may apply the composition to the skin then apply the article on top.

### ELECTROCHEMICAL REACTION

Generally, when it is sought to administer an active ingredient using the mask according to the invention, said active ingredient has the same polarity as the electrode. For example, compounds containing the active ingredients of positive charge/polarity such as vitamin A, tocopheryl acetate or other active ingredients of positive polarity/charge may be associated with an electrode of positive polarity.

Compounds containing active ingredients of negative charge/polarity such as retinyl palmitate, tocopherol, mandelic acid, and ascorbic acid may, for their part, be associated with an electrode of negative polarity.

The reaction may be an electro-osmosis.

### THE CARRIER

The carrier forms the matrix of the article.

### Dimensions and Structure

The carrier may, in the unfolded state, have a largest area comprised between 0.5 cm² and 500 cm², for example between 50 cm² to 300 cm².

The carrier may, in the unfolded state, have a thickness comprised between 0.05 cm and 1 cm and preferably between 0.1 cm and 0.5 cm.

### Material of the Carrier

Advantageously, the carrier comprises a woven or nonwoven material. The carrier is thus more supple. It better adapts to the contours of the chosen zone.

By “nonwoven” what is meant in the context of the present invention is a fiber-comprising substrate in which the individual fibers or filaments are arranged in a disordered manner in a structure taking the form of a web. The fibers of the nonwoven are generally bound to one another, either under the effect of a mechanical action, or under the effect of a thermal action, or by the addition of a binder.

Such a nonwoven is for example defined by standard ISO 9092.

The carrier may be occlusive and include sheets that are impermeable to water and/or to air. It may also include a treatment in order to make it occlusive.

### DESCRIPTION OF THE FIGURES

The invention will be better understood on reading the following description of nonlimiting implementations thereof and on examining the appended schematic and partial drawings, in which:

FIG. 1 shows alone a perspective front view of a double unitary block usable according to the invention,

FIG. 2 shows alone a perspective side view of the block of FIG. 1, only the button cells being shown at the front of the figure,

FIG. 3 is an axial cross section through a detail of embodiment of an article according to the invention equipped with a block according to FIG. 1,

FIG. 4 shows a front view of a first mask according to the invention said mask being equipped with a block according to FIG. 1,

FIG. 5 shows a front view of a second mask according to the invention, said mask being equipped with a block according to FIG. 1.

In the figures, for the sake of clarity, the various elements have not always been drawn to scale.

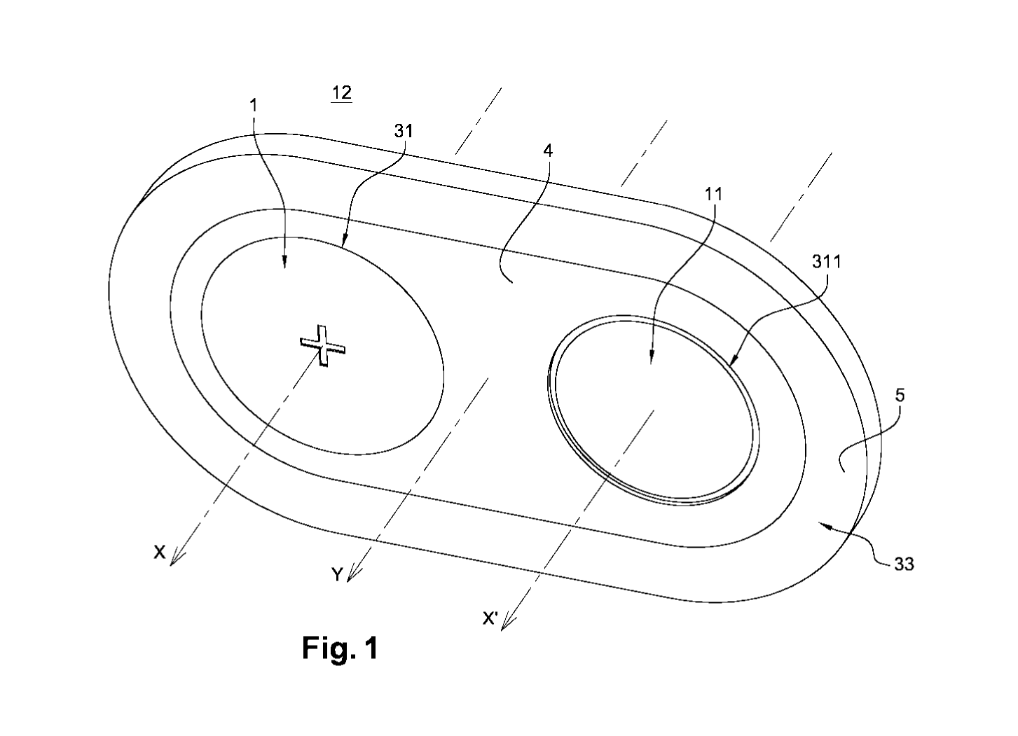

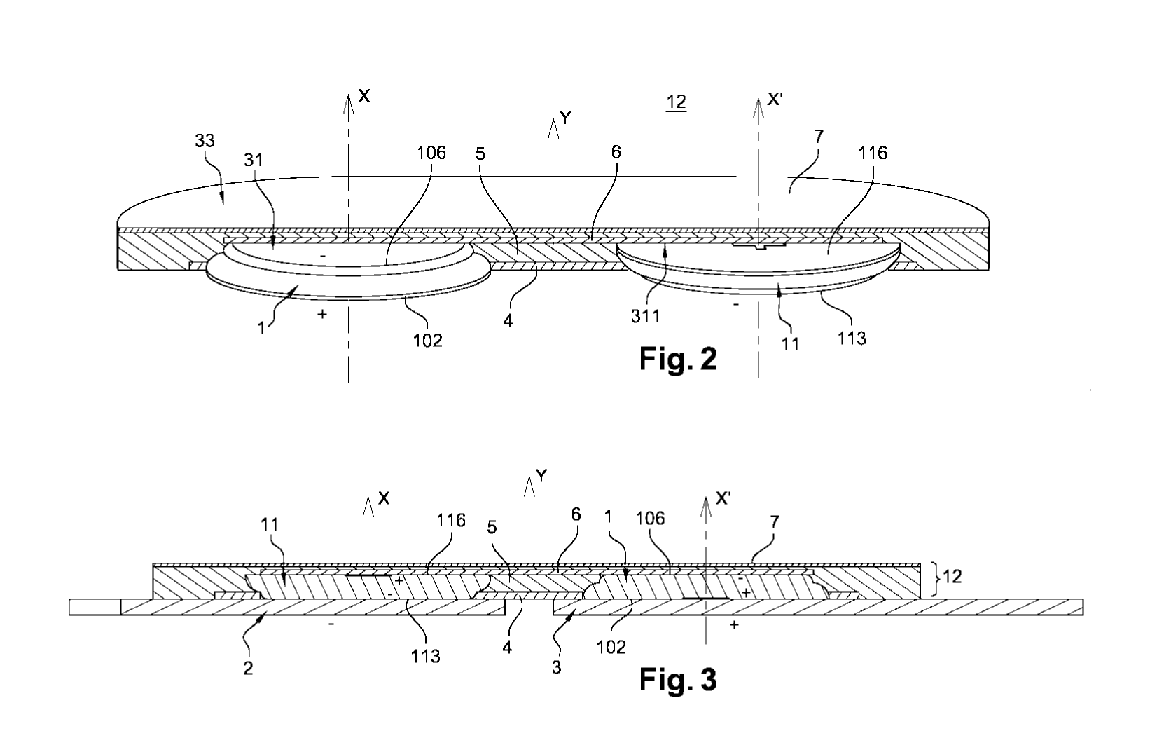

FIG. 1 and FIG. 2 show a unitary block 12 of axis Y.

The unitary block 12 mainly includes:
a source of electrical power formed by two button cells 1 and 11, and
a repositionable adhesive material 5 placed on a segment of the exterior surface 33 of the block 12.

Each battery 1 and 11 is flat. It has a dome shape of axis X (X′ respectively) the bottom of which forms:
the negative pole 106 for the battery 1,
the negative pole 113 for the battery 11.

The portion of the dome opposite the bottom with respect to the axis X (X′ respectively) forms:
the positive pole 102 for the battery 1,
the positive pole 116 for the battery 11.

Each battery 1 and 11 has a diameter comprised between 4 and 40 mm and a height comprised between 0.8 mm and 10 mm. The batteries 1 and 11 typically each have an operating voltage of more than 1 V, 3 V for example.

Preferably, a lithium button cell is used because it has a high discharge plateau and excellent discharge supply characteristics. The degree of utilization of the active material of the positive or negative electrode is considerably improved and the capacity of the battery is increased.

Button cells usable according to the invention are for example described in documents WO201310682 or WO2012000303.

The unitary block 12 includes two housings 31 and 311 that are intended to receive the battery 1 and the battery 11, respectively. These housings 31 and 311 are delineated by a repositionable adhesive 5, for example a silicone, sandwiched between a conductive sheet 6 and an insulating sheet 4. A flexible and resistant protective sheet 7 is fastened to the conductive sheet 6. This protective sheet 7 adheres to the repositionable adhesive 5.

FIG. 3 shows:
the positive pole 102 of the battery 1 making contact with a positive electrode 3,
the negative pole 106 of the battery 1 making contact with the conductive sheet 6,
the positive pole 116 of the battery 11 making contact with the conductive sheet 6,
a negative pole 113 of the battery 11 making contact with a negative electrode 2.

The adhesive flexible material 5 is insulating. It separates the positive pole 102 of the button cell 1 and the negative pole 113 of the button cell 11 from the sheet 6.

The insulating sheet 4 may include any electrically insulating material, for example chosen from insulating thermoplastic materials such as the polyamides, the styrenics, the polyolefins or the vinylics.

Each cell 1 and 11 delivers a current of 3 V for example, thereby making a sum of 6 V in total, making it possible to observe an effect of the treatment over a large area by the mask for example.

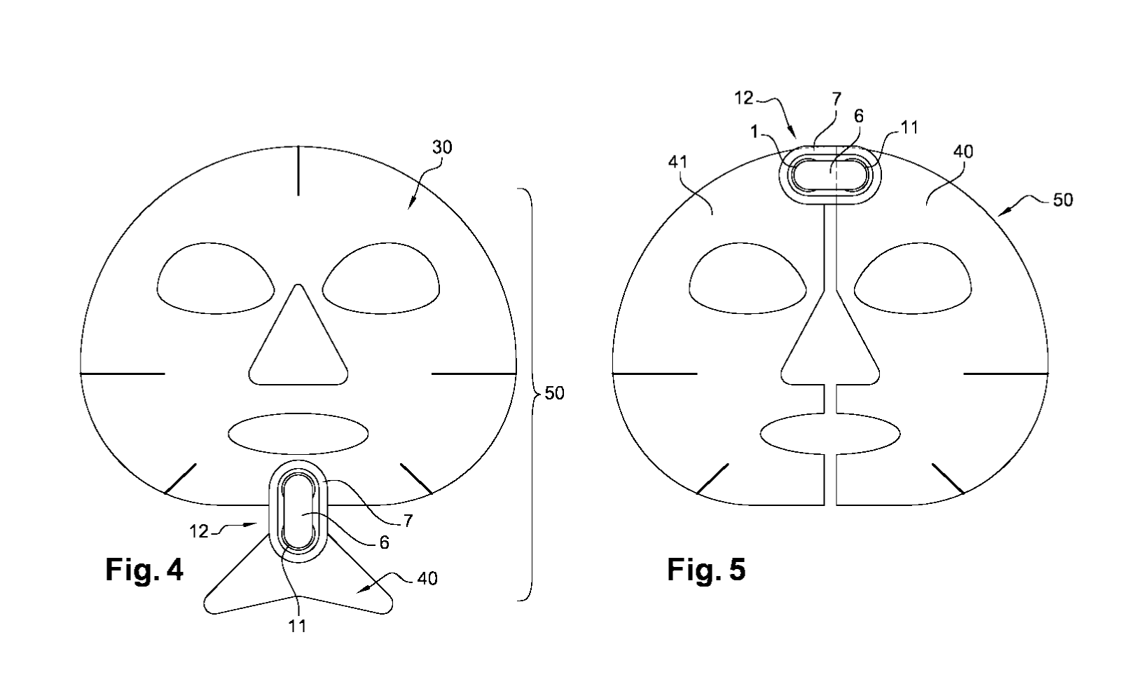

In FIG. 4 a mask has been shown that is configured to cover at least some of the face, and that includes a carrier 50 formed by a first portion 30 to be placed on the face and by an extension 40 to be placed under the chin.

The unitary block 12 is located between the first portion 30 and the extension 40.

It may be placed in a first direction with respect to the electrodes in order to provide care for the skin, and in a second direction in order to clean the skin.

The mask may, as shown in FIG. 5, include the unitary block 12 of FIG. 1 in the region of the forehead, and a carrier 50 formed of two portions: a right-hand portion 40 and a left-hand portion 41.

It is for example possible to connect:
a negative electrode 2 of the right-hand portion 40 to the (−) pole of the button cell 11, and
a positive electrode 3 of the left-hand portion 41 to the (+) pole of the button cell 1.

It is also possible to switch the electrodes in order to change treatment type as the block 12 is repositionable. Simply by flipping the unitary block 12 the treatment is changed from a skincare treatment to a skin-cleaning treatment.

As the mask is divided into two, the current applied to the face is increased and the effect of the treatment is improved.

The invention is not limited to the examples that have just been described.

Particularities of embodiment of the illustrated examples may be combined within variants that have not been illustrated. The structure of the electrodes and the area that they occupy may in particular be different. It is possible to switch the electrodes as the unitary block is repositionable. The article may be used in a penetration or stimulation mode. Other types of batteries or cells may be used.

1. A supple cosmetic article comprising at least one carrier (50) on which are placed at least one first pair of electrodes (2, 3) comprising a first electrode (2) and a first counter-electrode (3) and a unitary block (12) including at least one source of electrical power (1, 11), the unitary block (12) is connected without cable to the first pair of electrodes (2, 3) and the source of electrical power (1, 11) comprises a first terminal (102) that is connected to the first electrode (2) and a second terminal (113) that is connected to the first counter-electrode (3), characterized in that the unitary block (12) includes a repositionable adhesive material (5) on at least one segment of its exterior surface (33), said segment being located facing the first pair of electrodes (2, 3).

2. The article as claimed in claim 1, wherein the repositionable adhesive material (5) at least partially encircles the first terminal (102) and the second terminal (113).

3. The article as claimed in claim 1, wherein the repositionable adhesive material (5) is a viscoelastic silicone gel.

4. The article as claimed in claim 1, wherein the repositionable material (5) forms a layer having a thickness between one hundredth of a millimeter and 5 millimeters.

5. The article as claimed in claim 1, wherein the unitary block (12) is at least partially supple.

6. The article as claimed in claim 1, wherein the source of electrical power (1, 11) provides a potential difference higher than or equal to 1 V.

7. The article as claimed in claim 1, wherein the source of electrical power (1, 11) is a source of DC voltage.

8. The article as claimed in claim 1, wherein the source of electrical power (1, 11) comprises one or more button cells or one or more double button cells.

9. The article as claimed in claim 1, wherein the carrier (50) comprises an extension (40).

10. The article as claimed in claim 1, wherein the carrier (50) is impregnated with a composition.

11. The article as claimed in claim 1, wherein comprising a plurality of compartments each comprising a composition.

12. A cosmetic method comprising:
putting in place on a bodily zone, a first article, in order to carry out a first cosmetic treatment,
making a first electrical current flow within said first article thus put in place,
removing the unitary block (12),
repositioning the removed unitary block (12) on a second pair of electrodes (2, 3) forming part either of the first article or of a second article, in order to carry out a second cosmetic treatment,
making a second electrical current flow within said first or second article thus put in place.

13. The cosmetic method as claimed in claim 12, wherein the first cosmetic treatment is different from the second cosmetic treatment.

14. The cosmetic method as claimed in claim 12, wherein the first cosmetic treatment and the second cosmetic treatment are selected from the group consisting of a make-up treatment, a care treatment, a cleaning treatment and an extracting treatment.
